(19) United States
(12) Patent Application Publication
(10) Pub. No.: US 2014/0200439 A1
(43) Pub. Date: Jul. 17, 2014

(54) METHODS OF ORIENTING AND REGISTERING DURING A SURGICAL PROCEDURE

(71) Applicant: The Cleveland Clinic Foundation, Cleveland, OH (US)

(72) Inventors: Wael K. Barsoum, Bay Village, OH (US); Jason A. Bryan, Avon Lake, OH (US)

(73) Assignee: The Cleveland Clinic Foundation, Cleveland, OH (US)

(21) Appl. No.: 14/147,684

(22) Filed: Jan. 6, 2014

Related U.S. Application Data
(60) Provisional application No. 61/751,397, filed on Jan. 11, 2013.

(51) Int. Cl. A61B 5/06

(52) U.S. Cl. CPC A61B 5/061
USPC 600/424

(57) ABSTRACT

A patient-specific instrument is placed into a predetermined setting position with respect to a patient tissue. A navigation aid is associated in a predetermined origin position with respect to the patient tissue. The navigation aid is zeroed while in the predetermined origin position to record the location and orientation of the navigation aid relative to the patient tissue. An instantaneous position of the zeroed navigation aid is determined with respect to the patient tissue. Feedback is provided to a user on a physical motion needed to bring the instantaneous position of the navigation aid closer to a predetermined desired target position. An indication is made when the instantaneous position of the navigation aid is sufficiently similar to the predetermined desired target position.

### RELATED APPLICATION

This application claims priority from U.S. Provisional Application No. 61/751,397, filed 11 Jan. 2013, the subject matter of which is incorporated herein by reference in its entirety.

### TECHNICAL FIELD

The present invention relates to an apparatus and method for use of an orientation scheme and, more particularly, to orienting and/or registering a frame of reference or other location indication relative to a patient tissue during a surgical procedure.

### BACKGROUND OF THE INVENTION

During a surgical procedure, it may be helpful for a surgeon to be able to orient, duplicate, register, or otherwise observe and/or dictate a trajectory, location, and/or position of a structure relative to a patient tissue, using a local (with respect to the patient) or global (with respect to the operating room environment or some tool/structure therein) frame of reference. For example, during “robotic” or other surgical-navigation related procedures, it may be desirable to fairly precisely register the patient's location and orientation relative to the base (or other “origin point”) of the robotic device. As another example, it may desirable in both surgical-navigation or otherwise-guided procedures to know the position and/or orientation of a tool or other structure/device in relation to a relatively stationary (i.e., one which does not substantially change position intra-surgically) patient tissue structure.

### SUMMARY OF THE INVENTION

In an embodiment of the present invention, a method of duplicating a location and an orientation relative to a patient tissue during a surgical procedure is described. A patient-specific instrument is placed into a predetermined setting position with respect to the patient tissue. A navigation aid is associated in a predetermined origin position with respect to the patient tissue via a physical relationship between the navigation aid and the patient-specific instrument. The navigation aid is zeroed while in the predetermined origin position to record the location and orientation of the navigation aid relative to the patient tissue. The zeroed navigation aid is removed from the patient-specific instrument. The patient-specific instrument is removed from the predetermined setting position. An instantaneous position of the zeroed navigation aid is determined with respect to the patient tissue. The instantaneous position of the zeroed navigation aid is compared to a predetermined desired target position. Feedback is provided to a user on a physical motion needed to bring the instantaneous position of the navigation aid closer to the predetermined desired target position. An indication is made when the instantaneous position of the navigation aid is sufficiently similar to the predetermined desired target position, such that the navigation aid achieves the predetermined desired target position with respect to the patient tissue by user manipulation without use of the patient-specific instrument during the comparison of the instantaneous position of the zeroed navigation aid to the predetermined desired target position.

In an embodiment of the present invention, a method of registering a coordinate system relative to a patient tissue during a surgical procedure is provided. A model of the patient tissue is provided. A coordinate indicator is associated in a predetermined origin position with respect to the model of the patient tissue. The predetermined origin position of the coordinate indicator, while associated with the model of the patient tissue, is recorded with respect to a global location system. At least one predetermined model location point on the model of the patient tissue is recorded with respect to the global location system. At least one predetermined tissue location point on the patient tissue is recorded with respect to the global location system. Each predetermined tissue location point bears a substantial correspondence to a selected one of the predetermined model location points. The position of the model of the patient tissue with respect to the global location system is related to the position of the patient tissue with respect to the global location system by comparing the predetermined tissue location point(s) to at least one of: the predetermined origin position of the coordinate anchor and the corresponding predetermined model location point(s).

In an embodiment of the present invention, a method of registering a coordinate system relative to a patient tissue during a surgical procedure is provided. A coordinate indicator is associated in a predetermined origin position with respect to the patient tissue. The predetermined origin position of the coordinate indicator, while associated with the patient tissue, is recorded with respect to a global location system. The position of the patient tissue with respect to the global location system is determined based upon the configuration of the coordinate indicator, when positioned in the predetermined origin position, with respect to the global location system.

### BRIEF DESCRIPTION OF THE DRAWINGS

For a better understanding of the invention, reference may be made to the accompanying drawings, in which:

FIG. 1 is a top schematic view of an example use environment for the present invention;

FIG. 2 is a top schematic view of one embodiment of the present invention;

FIG. 3 is a side schematic view of the embodiment of FIG. 2;

FIG. 4 is a top schematic view of the embodiment of FIG. 2;

FIG. 5 is a side schematic view of another embodiment of the present invention;

FIG. 6 is a top schematic view of the embodiment of FIG. 5; and

FIG. 7 is a side schematic view of the embodiment of FIG. 5.

### DESCRIPTION OF EMBODIMENTS

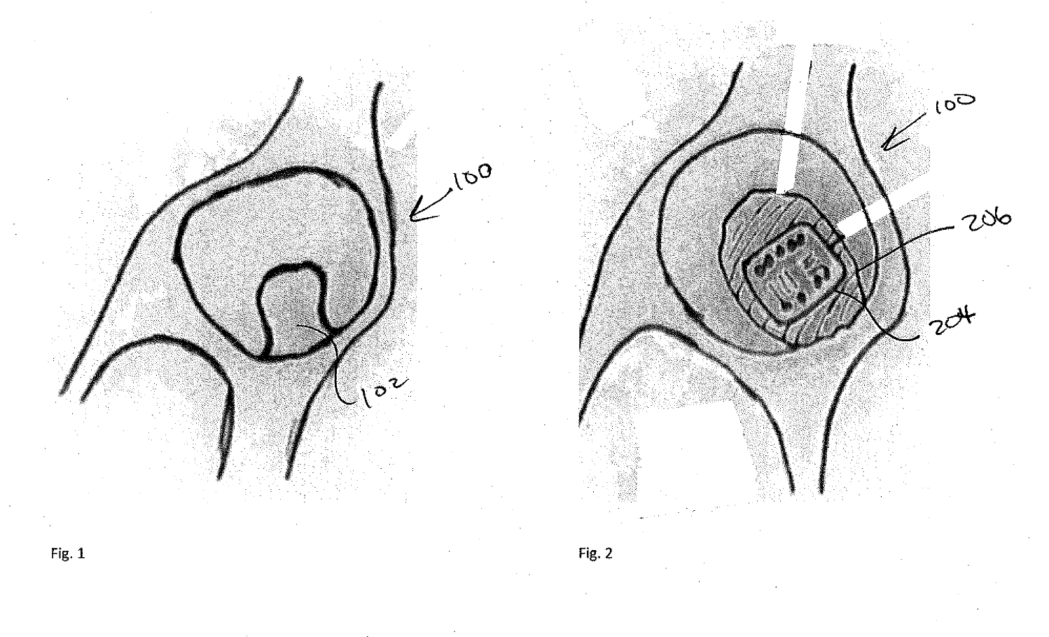

In accordance with the present invention, FIG. 1 depicts a top schematic view of an acetabulum 100 of a patient, with the fossa 102 prominently visible. The patient tissue shown and described herein is an acetabulum 100, but the patient tissue could be any desired types such as, but not limited to, hip joints, shoulder joints, knee joints, ankle joints, phalangeal joints, metatarsal joints, spinal structures, long bones (e.g., fracture sites), or any other suitable patient tissue use environment for the present invention. The below description presumes that the system, apparatus, and method described is being used in conjunction with a surgical procedure (namely, an at-least-partial joint replacement or resurfacing), but the system, apparatus, and method described may be used in any desired manner and for any desired purpose without harm to the present invention.

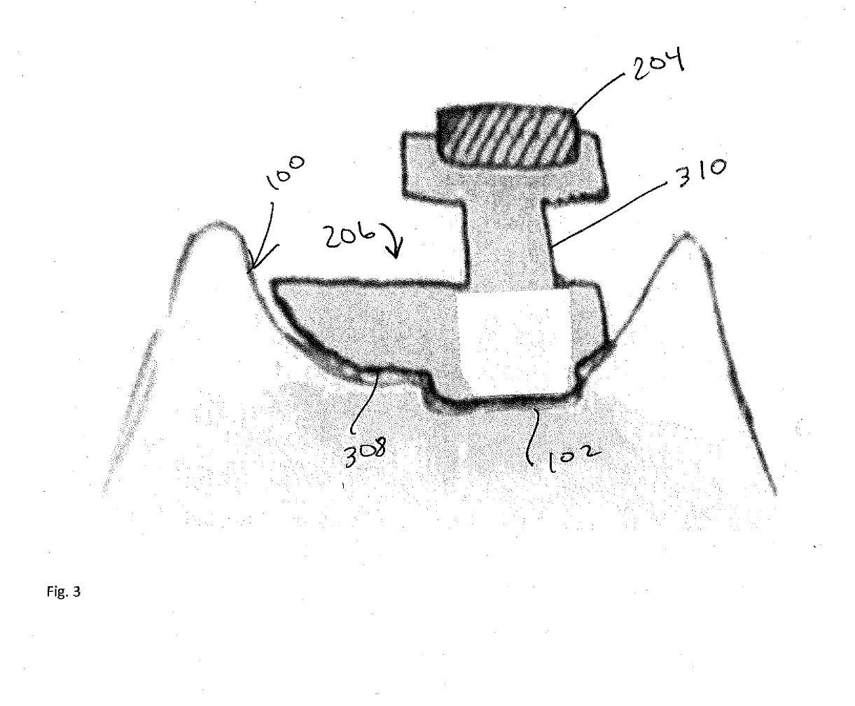

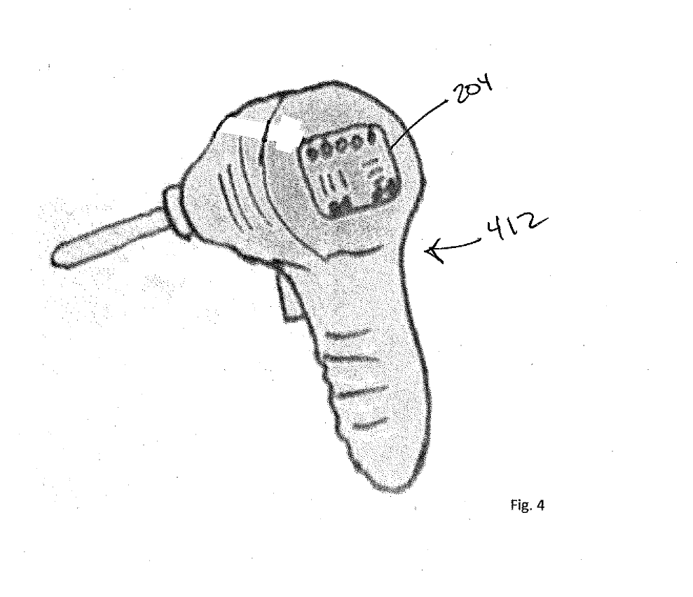

In FIG. 2, a navigation aid 204 has been placed in a predetermined origin position with respect to the acetabulum 100. As can be seen in FIGS. 2 and 3, the predetermined origin position of the navigation aid 204 is dictated by a physical relationship between the navigation aid and a patient-specific instrument (“PSI”) 206 that has been placed into a predetermined setting position with the acetabulum 100. The PSI could be any desired type, such as, but not limited to, those described in U.S. patent application Ser. No. 13/282,509, titled “System and Method for Association of a Guiding Aid with a Patient Tissue” and filed 27 Oct. 2011, the entire contents of which are incorporated herein by reference. The PSI could also or instead include adjustable, reusable features of any desired type, such as, but not limited to, those described in U.S. patent application Ser. No. 13/733,346, titled “Apparatus and Method for Dictating At Least One of a Desired Location and a Desired Trajectory for Association of a Landmark with a Patient Tissue” and filed 3 Jan. 2013, the entire contents of which are also incorporated herein by reference.

The PSI 206 holds the navigation aid 204 in the predetermined setting position through the PSI's own physical configuration. For example, and as shown in FIG. 3, the PSI 206 includes a tissue-contacting surface 308 which is configured patient-specifically to nest or mate in only one “unique” orientation with the acetabulum 100 (e.g., through a matched contour to that of at least the fossa 102 of the acetabulum). A stem 310 holds the navigation aid 204 at a predetermined elevation above the surface of the acetabulum 100. The navigation aid 204 therefore is held in a single predetermined setting position with respect to a particular acetabulum 100 (or other patient tissue, as appropriate) through configuration of the PSI 206 and the physical relationship between the navigation aid, the patient tissue, and the PSI.

Once the navigation aid 204 is held in the predetermined origin position by the PSI 206 as desired, the navigation aid is zeroed to record its location and orientation relative to the patient tissue at the predetermined origin position. The term “zeroed” is used herein to indicate that a point of departure in 3-dimensional reckoning is recorded, in any suitable manner, on the navigation aid 204. For example, the user could press a “tare” or “zero” button on the navigation aid 204 to indicate that the navigation aid was, at that time, in the predetermined origin position.

The navigation aid 204 includes and/or is operatively connected to (wired and/or wirelessly) sufficient sensors (e.g., accelerometers, magnetometers, high-resolution integrators, and/or GPS sensors), processors, user interfaces, and other components as desired to provide the functions described herein. Included among those components may be sensors of sufficient precision to provide location/orientation indications having useful resolution, with an acceptable amount of “drift”, for the purposes of the surgical assistance described herein. For example, the navigation aid 204 shown in FIGS. 1-4 may be used in conjunction with a “wireless” surgical navigation system that uses local or relative coordinates at the surgical site interface, rather than a traditional articulating armature surgical navigation system which orients with respect to the entire operating room and/or a navigation base unit, separate from the patient. Through use of the PSI 206, the navigation aid 204 can be locally registered to the patient tissue at the surgical site without requiring the user to touch or “key off” specific preselected “landmark” areas on the patient tissue, as is done in traditional locally registered surgical navigation systems. Because it can be difficult for a user to precisely eyeball or estimate a preselected landmark location on the patient tissue in some surgical exposures, the PSI 206 therefore may add to the repeatability and/or precision of the registration process.

Once the navigation aid 204 has been zeroed, it may be removed from the PSI 206 and the PSI may be removed from the predetermined setting position (e.g., the PSI may be removed from the patient tissue and set aside for later use or disposal). Alternately, and particularly if the PSI 206 is configured for mating with a non-target portion of the patient tissue (which is not coincident with the portion of the patient tissue which will be altered/adjusted or otherwise affected by the surgical procedure), the PSI could stay in the predetermined setting position during the surgical procedure.

It is also contemplated that one or more additional PSIs and/or additional navigation aids (not shown) could be provided for any desired purpose related to orientation and/or registration. For example, an additional navigation aid could be maintained in a relatively stationary position relative to the patient tissue, such as via an additional PSI and/or attachment directly to patient tissue. In this manner, movement of the patient tissue during a surgical procedure can be tracked via the additional navigation aid and compensated for in use of the main/primary PSI 206 and the associated main/primary navigation aid 204.

Regardless of the number, type, and location of PSIs and additional navigation aids provided, though, the zeroed navigation aid 204 enters a tracking/guiding mode after the zeroing function is completed. The sensors of the zeroed navigation aid 204 detect physical movement of the navigation aid from the predetermined origin position. For example, if the zeroed navigation aid 204 is placed on a table beside the patient, the navigation aid may “know” that it has then achieved a location and orientation which is +12.2 cm in the Z direction, −82.7 cm in the Y direction, and +17.4 cm in the X direction from the “zero” or “origin” location—i.e., the predetermined origin position. The resolution, precision, and other properties of the sensors, processing system, or other components of the navigation aid 204 or related technologies should be chosen to provide sufficient X, Y, Z, roll, pitch, yaw, or any other location/orientation indications for desired usefulness in the surgical procedure.

The instantaneous position of the zeroed navigation aid 204 relative to the patient tissue (here, the acetabulum 100) is determined and compared to the predetermined origin position in order to place the zeroed navigation aid into a predetermined desired target position, which is not necessarily the same as the predetermined origin position. This determination and comparison should be carried out and repeated with sufficient frequency and precision for the use environment/application of the present invention. The zeroed navigation aid 204, or a separate display or user interface, is then used to provide feedback to the user on a physical motion which is needed to bring the instantaneous position of the zeroed navigation aid 204 closer to the predetermined desired target position. For example, a “bubble level” or “bullseye” visual indication, a numerical +/− scale, an increasing pitch/frequency audible indication, or any other form of feedback could be used. The feedback may include instructions to the user to move the zeroed navigation aid 204 (and any associated surgical tool/aid) away from the instantaneous position in any direction, rotate (tilt/pan/etc.) the zeroed navigation aid without substantial translational X/Y/Z movement, and/or maintain the zeroed navigation aid in the instantaneous position.

Once the instantaneous position of the zeroed navigation aid 204 has become sufficiently similar to the predetermined desired target position to indicate, within the desired accuracy/precision, that the navigation aid has achieved the predetermined desired target position with respect to the patient tissue, an indication is given to the user. In this manner, the location and orientation of the zeroed navigation aid 204 in the predetermined desired target position can be achieved by user manipulation without use of the PSI 206 during the comparison of the instantaneous position of the zeroed navigation aid to the predetermined desired target position.

On its own, this ability to achieve the predetermined desired target position (whether or not the same as the predetermined origin position) of the location and orientation of the navigation aid 204 may have certain usefulness during surgical procedures. However, it is also contemplated that the navigation aid 204 could be associated with (e.g., mounted on) at least one surgical device 412 (a drill is shown in FIG. 4) in order that the location and orientation of the surgical device relative to the patient tissue can be relatively precisely controlled and/or reproduced. (The term “surgical device” is used herein to indicate any tool, prosthesis, natural or artificial patient tissue, or any other structure, device, or component for which it is desired to know and/or dictate a location and/or orientation with respect to a patient tissue.) The PSI 206 and/or navigation aid 204 could be configured with an “offset” value to account for the physical dimensions of a particular surgical device 412 and a particular predetermined desired target position, optionally with respect to the predetermined origin position. For example, the PSI 206 and/or navigation aid 204 could be used to position a drill bit in a predetermined orientation, then the drill could be actuated to drill into the patient tissue, still under guidance of the navigation aid, so that the end result, with the navigation aid 204 in the predetermined desired target position, is the drill bit penetrating a predetermined depth into the patient tissue at a predetermined trajectory/orientation.

It is also contemplated that different offsets could be determined or provided for a series of surgical devices 412 used sequentially during the surgical procedure. For example, once the zeroed navigation aid 204 has been placed into a known physical relationship with a reaming instrument (not shown), a first predetermined desired target position can correspond to a desired position and orientation of this reamer after the patient tissue is reamed to a desired depth for placement of an acetabular implant. The navigation aid 204, and/or some other portion of the related surgical navigation system will already be adjusted to account for the offset between the predetermined origin position and a reamer-associated predetermined desired target position. The zeroed navigation aid 204 can then be removed from the reamer and placed into an impactor (not shown) in a known physical relationship. The surgical navigation system can then be alerted that the zeroed navigation aid 204 has been moved from the reamer to the impactor and a second (impactor) predetermined desired target position—different from the first (reamer) predetermined desired target position—could then be used as a renewed target for adjustment of the zeroed navigation aid.

As another example, the navigation aid 204 and/or an associated surgical navigation system “knows” that it needs to orient the reamer and impactor for installation of an acetabular cup at 20° anteversion and 40° abduction. The navigation aid 204 can sense its position/orientation in space (i.e., absolutely, with respect to the ground). The navigation aid 204 has one “tare/zero” button which is actuated to tell the navigation aid that it is oriented at 0° anteversion and 0° abduction with respect to the patient tissue (here, a pelvis). The PSI 206 is configured to hold the navigation aid 204 at a position in space that the user wants to equate to such a “0/0” measurement—this is the predetermined origin position. Once it is held in the predetermined origin position by the PSI 206, the navigation aid 204 is zeroed (e.g., through depression of a “tare” button). The navigation aid 204 then “remembers” its internal state at the predetermined origin position and, when moved from the predetermined origin position, the navigation aid (or another component of the surgical navigation system) can very quickly compare the instantaneous state of the navigation aid to its prior state in the predetermined origin position to determine how far from 0/0 the navigation aid is located. The navigation aid 204 may then be placed into a preconfigured holder on a reamer, impactor, or other surgical device 412. The navigation aid 204 then reports its current instantaneous angles to the surgical navigation system or directly to a user, who can move the instrument until the navigation aid achieves a “20/40” anteversion/abduction position (that 20/40 being a predetermined desired target position, evaluated with respect to the original zero location, a.k.a. the predetermined origin position). The user will then know that the desired orientation/location for the surgical device 412 has been achieved, and the surgical procedure may proceed accordingly.

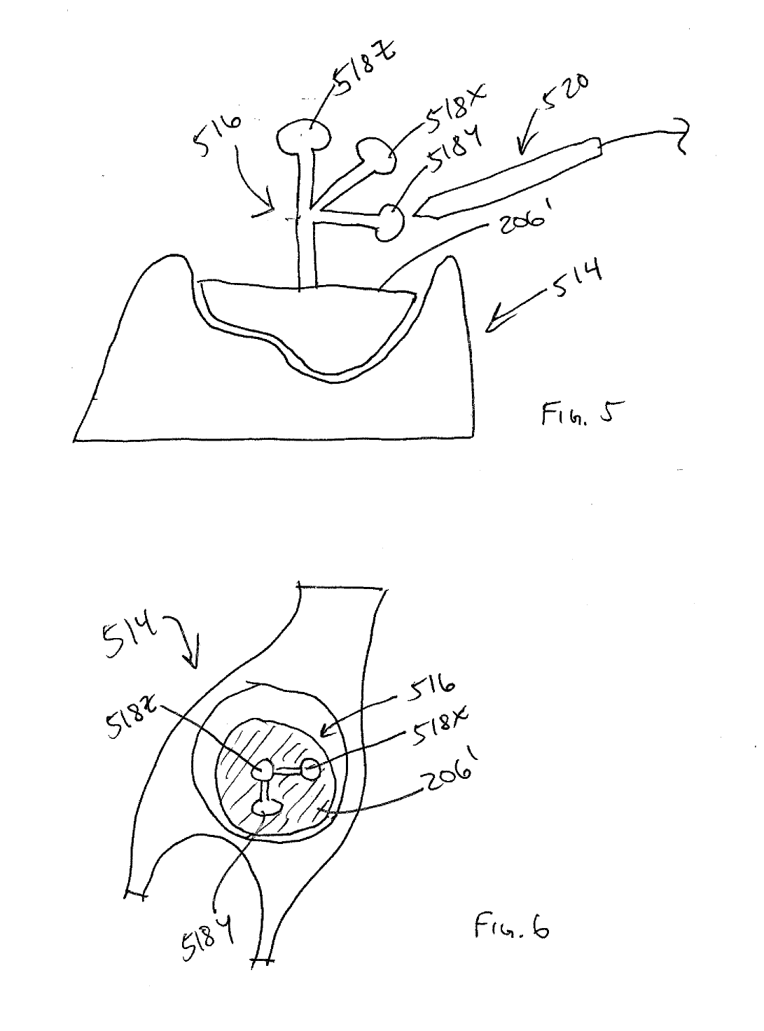

FIG. 5 depicts a physical configuration for assisting with a method of registering a coordinate system relative to a patient tissue (again, an acetabulum 100) according to another embodiment of the present invention. The embodiment shown in FIGS. 4-7 may be useful, for example, in a broad-area surgical navigation system that uses an “absolute” positioning system with respect to the operating room, a base unit, or some other non-patient reference/origin point--in contrast to the local positioning system described above with reference to the previous embodiment of FIGS. 1-4.

In FIG. 5, a model 514 of the native patient tissue of an acetabulum 100 is provided. The term “native patient tissue” and variants thereof is used herein to indicate a patient tissue of interest in its condition at the time of surgical preparation, having any included natural or artificial structures of interest, whether congenital or acquired. The term “model” is used herein to indicate a replica or copy of a physical item, at any relative scale and represented in any medium, physical or virtual. (However, in the below description, the model 514 will be presumed to be a physical model for reasons which will become apparent, as opposed to a virtual model located only on a computer system.) The patient tissue model 514 may be a total or partial model of a subject patient tissue, and may be created in any suitable manner. For example, and as presumed in the below description, the patient tissue model 514 may be a tangible representation of a virtual model generated using computer tomography (“CT”) data imported into a computer aided drafting (“CAD”) system. Additionally or alternatively, the patient tissue model 514 may be based upon a virtual model created with the aid of digital or analog radiography, magnetic resonance imaging, or any other suitable imaging means. The patient tissue model 514 will generally be displayed for the user to review and manipulate preoperatively, such as through the use of a physical model, as described herein.

The patient's name, identification number, surgeon's name, and/or any other desired identifier may be molded into, printed on, attached to, or otherwise associated with the physical model 514 in a legible manner. Particularly when based upon a virtual model, the physical model 514 may be made by any suitable method such as, but not limited to, selective laser sintering (“SLS”), fused deposition modeling (“FDM”), stereolithography (“SLA”), laminated object manufacturing (“LOM”), electron beam melting (“EBM”), 3-dimensional printing (“3DP”), contour milling, computer numeric control (“CNC”), other rapid prototyping methods, or any other desired manufacturing process. An example of a suitable model 514 is provided in U.S. patent application Ser. No. 13/282,550, titled “System of Preoperative Planning and Provision of Patient-Specific Surgical Aids” and filed 27 Oct. 2011, the entire contents of which are incorporated herein by reference.

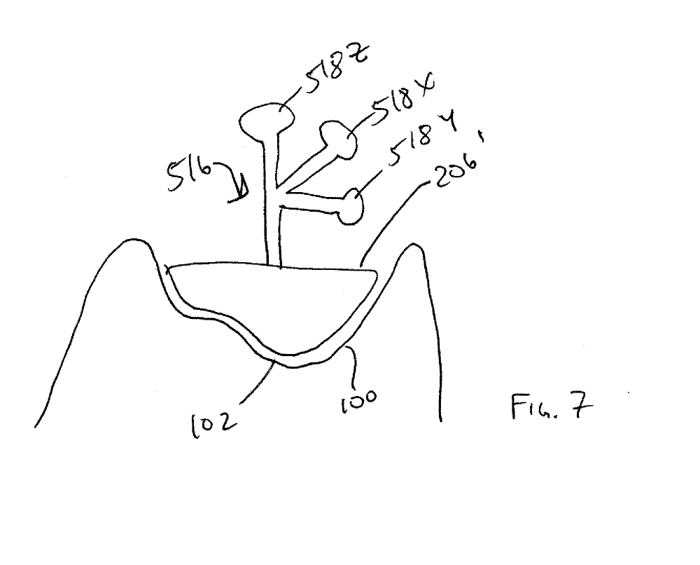

As shown in FIG. 5, a PSI 206′, which can only mate with the model 514 in one orientation, carries a coordinate indicator 516. The coordinate indicator 516 includes orthogonal arms 518X, 518Y, and 518Z corresponding to a coordinate origin. Because of the physical relationship between the PSI 206′ and the model 514, the coordinate indicator 516 becomes associated with the model of the patient tissue in a predetermined origin position, as shown in side view in FIG. 5 and in top view in FIG. 6.

As shown in FIG. 5, a wand 520 associated with a surgical navigation system may be used to contact each of the orthogonal arms 518X, 518Y, and 518Z in turn to record their position with respect to a global location system while the coordinate indicator 516 is in the predetermined origin position with respect to the model 514. Here, though the wand 520 is described as “touching” or “contacting” the target areas, it is also contemplated that no actual physical contact may need be made for the wand to still perform the functions herein to record the three-dimensional absolute and/or relative positions of the target areas as desired. One of ordinary skill in the art could readily provide a suitable registration scheme in the context of the present invention for a particular use environment.

For example, the model 514 could be sitting on a table (perhaps in a holding jig) in a surgical navigation-equipped operating room so that the model is relatively fixed in position in the room. The wand 520 is therefore used to positively indicate to the surgical navigation system, through use of the PSI 206′ and the coordinate indicator 516, the absolute position of the model 514 within the room. The wand 520 can then be used to touch at least one preselected landmark area (i.e., a predetermined model location point) on the model 514, which then informs the surgical navigation system of the orientation and location of the model within the room. For example, a series of virtual predetermined model location points could be sequentially illuminated on a computer screen view of a virtual model, and the user responsively touches the wand 520 to the same predetermined model location points on the model 514 when prompted. This registration scheme may be particularly useful and accurate when the predetermined model location points are chosen to be relatively easy to identify and access, such as relatively small protrusions, notches, or other small-scale and relatively easy-to-locate features of the patient tissue. As an alternative to those preselected landmark areas, the user could choose/predetermine her own predetermined model location points in real-time during the registration procedure by touching the wand 520 to the model 514; because the surgical navigation system “knows” the relationship of the patient tissue to the coordinate indicator 516 and the position of the coordinate indicator 516 within the room, the surgical navigation system will then be able to store those user-chosen predetermined model location points as “landmarks” on the virtual model.

As a first option for proceeding, the PSI 206′ and associated coordinate indicator 516 may then be removed from the model 514 and placed into the predetermined origin position with respect to the actual patient tissue--here, the acetabulum 100 shown in FIG. 7. Because the PSI 206′ fits onto both the model 514 and the actual patient tissue in substantially the same unique orientation, the predetermined origin position of the coordinate indicator 516 with respect to the actual patient tissue acetabulum 100 will be substantially similar to the predetermined origin position of the coordinate indicator with respect to the model 514.

The wand 520, or another similar device, may then again be touched (or otherwise activated) to each of the orthogonal arms 518X, 518Y, and 518Z in turn to record their position with respect to the global location system in this first option while the coordinate indicator 516 is in the predetermined origin position with respect to the patient's acetabulum 100. The wand 520 is therefore used to positively indicate to the surgical navigation system, through use of the PSI 206′ and the coordinate indicator 516, the absolute position of the actual patient tissue within the room, as well as the relative position/orientation of the patient tissue as compared to the model 514. It is contemplated that both the model 514 and the actual patient tissue (e.g., acetabulum 100) will be relatively rigidly affixed in their respective absolute positions within the room such that the global location system considers them “fixed” in position within the surgical navigation space.

Additionally or alternatively, the user could cease use of the PSI 206′ once the model 514, and the predetermined model location points, have been registered with the global location system. In such event, the PSI-less acetabulum 100 or other patient tissue could include at least one predetermined tissue location point. Each predetermined tissue location point bears a substantial correspondence to a selected one of the predetermined model location points which have already been established/oriented with use of the model 514 (whether as preselected landmark areas or real-time user-selected portions of the model). In other words, if the model 514 were three-dimensionally superimposed on the corresponding patient tissue, the position of a particular predetermined tissue location point on the patient tissue would be sufficiently similar to the position of a corresponding predetermined model location point for a desired correlation between the two to support a surgical navigation scheme of a desired precision and accuracy.

In the PSI-less patient tissue option, then, the user touches the wand 520 to the predetermined tissue location points on the patient tissue, optionally guided by sequential highlighting of similar landmark areas on a virtual model of the patient tissue. The surgical navigation system will then “know” the location/orientation of the patient tissue, in an absolute sense, with respect to the global location system, and will also “know” the location/orientation of the corresponding model 514 with respect to the global location system. By extrapolation from the model 514 assisted registration process, the (global) position of the patient tissue within the room can then be related to the (global) position of the model 514 within the room, as can their corresponding local coordinate systems as embodied in the PSI 206′.

Once the global location system has recorded the absolute positions/locations/orientations of the model 514 and the patient tissue, these positions can be related to one another and the relative positions can be used during a surgical procedure. For example, if a user places a landmark (e.g., a guide pin) in a desired trajectory/location with respect to the model 514 (e.g., to indicate a desired drilling trajectory/location), the global location system, optionally with the assistance of a surgical navigation system, can be used to transfer or duplicate the trajectory/location with respect to the actual patient tissue.

It is contemplated that, in the embodiment of FIGS. 5-7, the PSI 206′, model 214, and/or the coordinate indicator 514 could be integrally provided as a one-piece unit (that is, functioning as a single structure during the process described above), particularly when the PSI-less registration scheme is used.

As a second option for the embodiment of FIGS. 5-7 with use of the PSI 206′, the model 514 could be omitted from this system. In such an arrangement, a PSI 206′ including a coordinate indicator 514 could be initially placed directly into a predetermined origin position with a patient tissue. The wand 520 can then be used in conjunction with the coordinate indicator 514 to “teach” the surgical navigation system the absolute position of the coordinate indicator, and hence the associated PSI 206′, with respect to the room. By extrapolation, the surgical navigation system can then determine the (global) position of the patient tissue with respect to the room based upon the known configuration of the coordinate indicator 514, which bears a known relationship to the PSI 206′, which, in turn and when in the predetermined origin position, bears a known relationship to the patient tissue.

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the navigation aid 204 or coordinate indicator 516 are merely illustrative; one of ordinary skill in the art could readily determine any number of tools, sequences of steps, or other means/options for placing the above-described apparatus, or components thereof, into positions substantively similar to those shown and described herein. The zeroed navigation aid 204 could include a low power “sleep” mode (automatically or manually induced) which conserves battery power if the zeroed navigation aid is not moved for a predetermined period of time. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications of the present invention. Though certain components described herein are shown as having specific geometric shapes, all structures of the present invention may have any suitable shapes, sizes, configurations, relative relationships, cross-sectional areas, or any other physical characteristics as desirable for a particular application of the present invention. Unless otherwise specifically stated, contact could be either direct or indirect. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. A method of duplicating a location and an orientation relative to a patient tissue during a surgical procedure, the method comprising the steps of:
placing a patient-specific instrument into a predetermined setting position with respect to the patient tissue;
associating a navigation aid in a predetermined origin position with respect to the patient tissue via a physical relationship between the navigation aid and the patient-specific instrument;
zeroing the navigation aid while in the predetermined origin position to record the location and orientation of the navigation aid relative to the patient tissue;
removing the zeroed navigation aid from the patient-specific instrument;
removing the patient-specific instrument from the predetermined setting position;
determining an instantaneous position of the zeroed navigation aid with respect to the patient tissue;
comparing the instantaneous position of the zeroed navigation aid to a predetermined desired target position;
providing feedback to a user on a physical motion needed to bring the instantaneous position of the navigation aid closer to the predetermined desired target position; and
indicating when the instantaneous position of the navigation aid is sufficiently similar to the predetermined desired target position, such that the navigation aid achieves the predetermined desired target position with respect to the patient tissue by user manipulation without use of the patient-specific instrument during the comparison of the instantaneous position of the zeroed navigation aid to the predetermined desired target position.

2. A method of registering a coordinate system relative to a patient tissue during a surgical procedure, the method comprising the steps of:
providing a model of the patient tissue;
associating a coordinate indicator in a predetermined origin position with respect to the model of the patient tissue;
recording the predetermined origin position of the coordinate indicator, while associated with the model of the patient tissue, with respect to a global location system;
recording at least one predetermined model location point on the model of the patient tissue with respect to the global location system;
recording at least one predetermined tissue location point on the patient tissue with respect to the global location system, each predetermined tissue location point bearing a substantial correspondence to a selected one of the predetermined model location points; and
relating the position of the model of the patient tissue with respect to the global location system to the position of the patient tissue with respect to the global location system by comparing the predetermined tissue location point(s) to at least one of: the predetermined origin position of the coordinate anchor and the corresponding predetermined model location point(s).

3. A method of registering a coordinate system relative to a patient tissue during a surgical procedure, the method comprising the steps of:
associating a coordinate indicator in a predetermined origin position with respect to the patient tissue;
recording the predetermined origin position of the coordinate indicator, while associated with the patient tissue, with respect to a global location system; and
determining the position of the patient tissue with respect to the global location system based upon the configuration of the coordinate indicator, when positioned in the predetermined origin position, with respect to the global location system.
